(19) United States
(12) Patent Application Publication
(10) Pub. No.: US 2017/0282850 A1
(43) Pub. Date: Oct. 5, 2017

(54) HEAD RESTRAINT APPARATUS WITH EXPANDABLE SEAT SECURING MECHANISMS

(71) Applicant: Diana M. Friedman, Evanston, IL (US)

(72) Inventor: Diana M. Friedman, Evanston, IL (US)

(21) Appl. No.: 15/478,896

(22) Filed: Apr. 4, 2017

Related U.S. Application Data
(60) Provisional application No. 62/317,957, filed on Apr. 4, 2016.

(51) Int. Cl. B60R 22/00; A61F 9/04; B60R 22/12

(52) U.S. Cl. CPC B60R 22/001; B60R 22/12; A61F 9/04

(57) ABSTRACT

An accessory device for use in conjunction with seats on airplanes, trains, buses, cars, or other moving vehicles that gently restrains a person's forehead to prevent his or her head from bobbing when he or she falls asleep in their seat. The device may have a head retention piece, which may also serve as a sleep mask, in addition to various configurations for securing the head retention piece to seats or the headrest portions of seats having various dimensions. In one embodiment, a base portion is secured to the seat and the head restraint piece secured to the base.

### FIELD OF THE INVENTION

The disclosure relates to a forehead restraint accessory for use while traveling on airplanes, trains, buses, cars and/or other moving vehicles.

### BACKGROUND OF THE INVENTION

When a person falls asleep in an upright seated position, particularly while sitting in the passenger seats of a moving vehicle, their head tends to bob forward or to the side. Such positions are uncomfortable and can often lead to frequent sleep interruption and sore muscles, particularly in the neck, shoulders and head. Accordingly, need exists for an apparatus or device which may be used with the passenger seats of commercial carriers which will prevent a person's head and neck from gravitating to one or more uncomfortable positions when asleep.

Accordingly, a need exists for an apparatus or device which may be used with the passenger seats of commercial carriers, such as airplane seats, train seats, bus seats, and car seats (“transportation seat”) which will prevent a person's head and neck from gravitating to one or more uncomfortable positions when an occupant is asleep in his seat.

In addition, many of these transportation seats vary in size, width, perimeter profile and upholstery. Others contain television or computer screens built into the back side thereof.

Accordingly, a further need exists for an apparatus or device which may be used with transportation seat which vary in size, width, perimeter profile, surface material upholstery and configuration, which will prevent a person's head and neck from gravitating to one or more uncomfortable positions when an occupant is asleep in his seat.

### SUMMARY OF THE INVENTION

Disclosed is an apparatus, herein referred to as the “NoBob”, for use in conjunction with seats on airplanes, trains, buses, cars, or other moving vehicles that gently restrains a person's forehead to prevent his or her head from bobbing when he or she falls asleep in their seat. As an additional benefit, the portion of the restraining device contacting the wearer's head may also serve as a darkening shade to enable the wearer to fall asleep more easily.

According to one aspect of the invention, the accessory device comprises a head retention piece and means for securing the head retention piece securely to the person's seat. In one embodiment, the accessory device comprises a head retention piece, portion of which is in contact with the wearer's head, and a pair of width adjustable clasps configurable for attaching to a transportation seat having any number of different width and profile configurations. One or more straps attaches the head retention piece to the clasps in a manner which retains the position of the retention piece on the wearer's head even during positional adjustments of the seat angle or slight movements by the wearer.

Since many of these transportation seats vary in size, width, and upholstery, the removable elastic strap is slip resistant and as such is securable to most seats in order to retain the accessory securely thereto. The head retention piece rests adjacent to the person's forehead and may optionally be decorated with a graphic, such as a sports team logo, or other graphic to increase the attractiveness of the accessory to of potential users of NoBob.

In one embodiment, a removable elastic strap is selectively engageable and disengagable at either side band of the head retention piece to form a loop. In some cases, seats have TV screens on the backs of these seats, and an elastic strap would disturb the occupant of the seat directly behind the occupant in the seat with the NoBob. As such the elastic strap needs to be removable and replaceable with two separate attaching mechanisms that attach securely to the sides of the occupant's seat without interfering with the occupant behind.

In one embodiment, the front retention cover is disposed at least partially about the front band and may be made to conform to the forehead and top of the head. This cover may also be disposed over at least a portion of the side bands. In one embodiment, the retention piece cover may be made of fleece and lining materials which are soft and comfortable against the person's forehead and scalp.

According to another aspect of the disclosure, a head restraint apparatus comprises a head retention piece, and a pair of width adjustable clasps secured to the head retention piece, each of the clasps being expandable to a width for temporarily secure the clasp about a surface having a thickness.

According to yet another aspect of the disclosure, a kit comprising an accessory device for use with transportation seats, comprises a head retention piece, a portion of which is in contact with the wearer's head, and at least one stretchable strap removably securable to the head retention piece and about a transportation seat.

According to still another aspect of the disclosure, a head restraint kit for use with transportation seats comprises a head retention piece, and a base portion adjustably securable to about a surface having a thickness and further temporarily securable to the head retention piece.

According to get another aspect of the disclosure, a method for securing a head restraint apparatus to a transportation seat, comprises providing a head restraint apparatus comprising a head retention piece having a pair of straps extending outward there from and a base portion adjustably securable about a surface having a thickness; removably securing the base portion to the surface; and removably securing the head restraint apparatus to the base portion.

### DESCRIPTION THE DRAWINGS

FIG. 1 illustrates conceptually a perspective view of an accessory device in accordance with the disclosure;

FIG. 2 illustrates conceptually a rear plan view of the accessory device FIG. 1 in accordance with the disclosure;

FIGS. 3A-B illustrate conceptually a plan views of the clasp component of the accessory device in an expanded and expanded configurations, respectively, in accordance with the disclosure;

FIGS. 4 illustrates conceptually a perspective view of the clasp component of FIGS. 3A-B in accordance with the disclosure;

FIGS. 5A-B illustrate conceptually a perspective view of another embodiment of the accessory device in accordance with the disclosure; and

FIGS. 6A-B illustrate conceptually a plan views of accessory sleep mask and headrest seat attachment device in accordance with the disclosure;

FIG. 7 illustrates conceptually a perspective view of the embodiment of FIGS. 6A-B as attached to the headrest of a seat.

### DETAILED DESCRIPTION

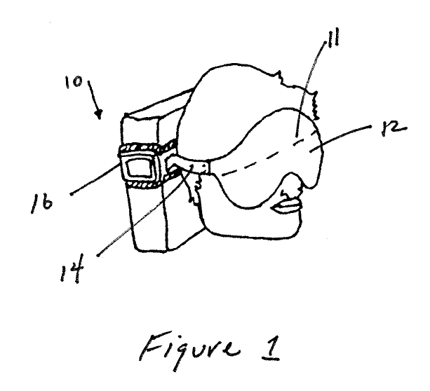

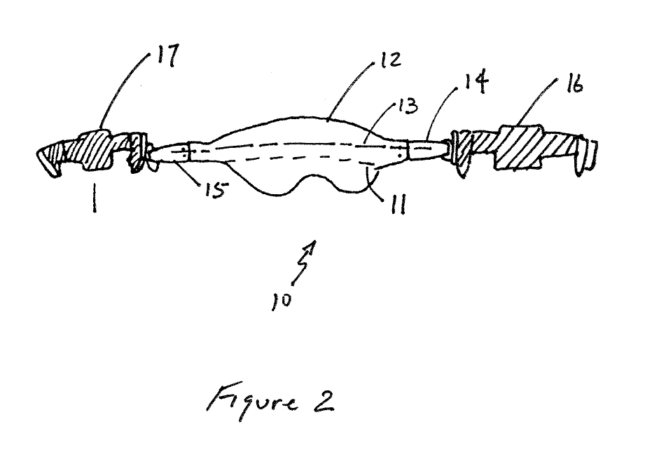

The NoBob is an accessory device that gently restrains a person's forehead to prevent his or her head from bobbing and rolling when falling asleep in a transportation seat. Referring to the FIGS. 1-2, an accessory device 10 comprises a head retention piece 12 having a pair of side bands 14 and 15 with one end secured thereto and extending in opposite directions outwardly therefrom. In one embodiment, side bands 14 and 15 extend along a common axis 13, illustrated in phantom in FIG. 2. Accessory device 10 further comprises a pair of expendable U-shaped brackets 16 and 17 attached to the other ends of side bands 14 and 15, respectively, through respective loops 18.

The head retention piece 12 may be made of fabric and shaped to accommodate the curvature of the forehead. In one embodiment, the head retention piece 12 may be made of fleece and/or lining materials which are soft and comfortable against the person's forehead and scalp. In one embodiment, head retention piece 12 may have a generally blindfold shape, as illustrated in FIG. 1, that is made of light blocking material to enable the wearer to more readily fall asleep. In one embodiment, the blindfold may have a partition 11 in the form of a seam extending at least partially across the width thereof to enable the wearer to flip the bottom portion of the blindfold up to see without having to remove the retention piece 12 completely.

A first end of each of side bands 14 and 15 may be either permanently or removably secured to head retention piece 12 by any of stitching, buckles, hook and pile connectors, snaps, Velcro, etc. In one embodiment, side bands 14 and 15 may be implemented with elastic, webbing or other durable fabric. In one embodiment, the other ends of side bands 14 and 15 may be movably or removably secured to U-shaped brackets 16 and 17, respectively, through the loop 18 or other mechanism to allow for angular movement of straps 14 and 15 relative to U-shaped brackets 16 and 17 one secured to a seat 19.

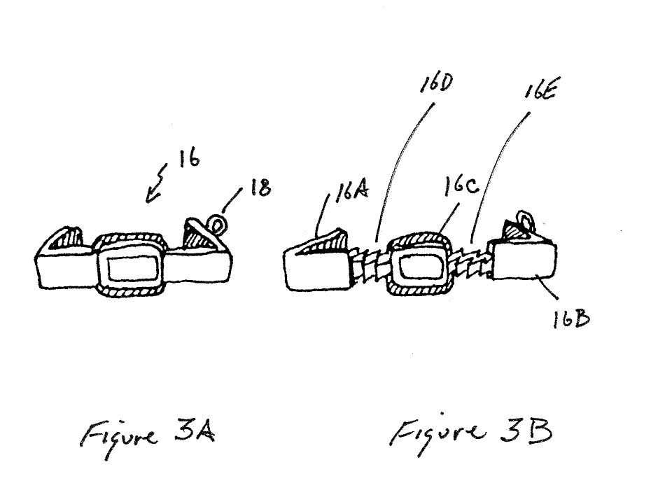

FIGS. 3A-B illustrate conceptually a plan views of the clasp component of the accessory device in an expanded and expanded configurations. Clasp 16 comprises a pair of substantially L-shaped rigid frames 16A and 166 expandably coupled to a center clasp mechanism 16C. Each of frames 16A and 16B may be formed of a substantially rigid material such plastic or other as natural or synthetic resins and may define along an interior surface there of a channel or groove (not shown). The back surface of each frames 16A and 16B may be covered with a soft fabric, padding, or nonslip material which has a high coefficient of friction, which will rest against seat 19. Disposed intermediate an interior portion of frames 16A and 16B are substantially ridged or ratcheted belts 16D and 16E which may be slidably extended outside of its respective. Belts 16D and 16E may define complementary mating, but oppositely oriented, teeth which may slide easily over each other when slightly separated therefrom, resulting in one or more adjacent engaged teeth as in a fully collapsed configuration, and, which when separated by a divider, such as a spring-loaded pin are separated wide enough to allow for movement in opposing directions to enable expansion of the ratcheted belts 16D and 16E so that only one set of teeth are engaged in an expanded position. Clasp mechanism 16C may define a frame through which ratcheted belts 16D and 16E may pass and a button 16F for selectively and pivotally introducing a dividing pin intermediate belts 16D and 16E to enable movement in opposite directions there to during expansion and collapsing thereof. Alternatively, any number of commercially available ratcheted locking devices, similar to those used in ski bindings and wire binding devices may be implemented in clasp mechanism 16 to achieve the same result.

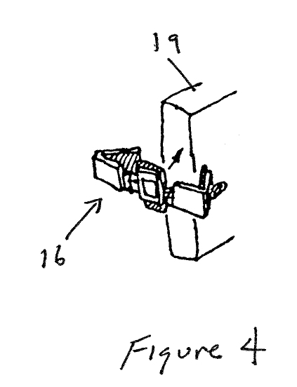

FIGS. 4 illustrates conceptually a perspective view of one of the clasp component 16 relative to the seat 19. In one embodiment, one of frames 16A and 16B may be longer than the other so that its respective point of attachment to strap 14 is closer to the interior of seat 19 thereby providing a more snug fit for retention piece 12 against the wearer's head and better light blockage. The construction and function of clasp 17 may be the same or different than clasp 16 as described herein.

In one embodiment, retention piece 12 may have a single continuous strap 22 which extends across the back of the wearer's head and is connectable to a loop strap 20 extending between loops 18 of clasps 16 and 17 via a loop through which loop strap 20 may be secured. In such a configuration, a single point of attachment may allow for slight head movement while continuing to restrain the wearer's close to seat 19.

All bands may comprise of a fabric band or a band having elastomeric characteristics such as elastic. The front band may comprise of a strap that extends from one side of the transportation seat, across the person's forehead, to the other side of the person's transportation seat and may be provided with adjustable clip buckles or Velcro at both ends. Side bands 14 and 15 may be connected to clasps 16 and 17, respectively, with loops 18, as illustrated, or any other attachment mechanism described herein. In addition, loops 18 may be at multiple different locations on either of frames 16A and 16B.

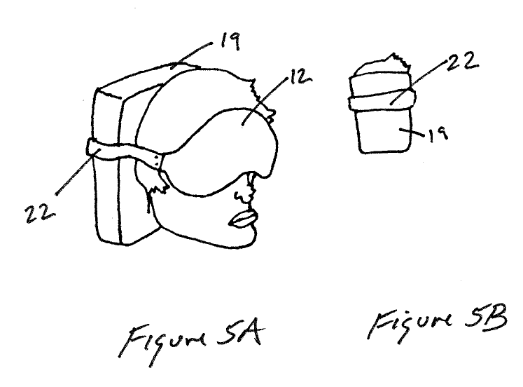

In another embodiment, as illustrated in FIGS. 5A-B, the back band is easily replaced by another set of bands that include or are attached to a mechanism that firmly attaches to the upper sides of the transportation seat. One form of this mechanism may be a clamp made out of plastic, metal, or other sturdy yet light material that has 3 sides, whereas one connecting side runs perpendicular to the other two sides with one side gripping the front seat and the other side gripping the back of the seat. Since transportation seats vary in width, the connecting side has a release mechanism for adjusting the length of the connecting side and locking into place once the clamp is snug against the front and back of the seat. In order to lessen the risk of slippage, the inside of the front and back sides of the mechanism may have a slip resistant material such as rubber or maybe ridged to ensure the clamps stay in place. This mechanism can take other forms as long as it can slide over the sides of the transportation seats locking into place and hold the side bands in place without interfering with either a TV or tray that is attached to the back of the occupant's seat.

In one embodiment, the side bands 14 and 15 may be removably secured to a single continuous band 22 having a selectively adjustable length and which is securable directly about seat 19. Alternatively, accessory device 10 may be provided as a kit with multiple bands 22 of various diameters which may be used to selectively fit around various seat configurations into which side bands 14 and 15 may be secured. In one embodiment, the side bands 14 and 15 may be replaced by the single continuous band 22 having a selectively extendable length and is securable directly to retention piece 12 about the back of seat 19. In embodiments, any configuration of continuous band 22 may be either stretchable or nonstretchable.

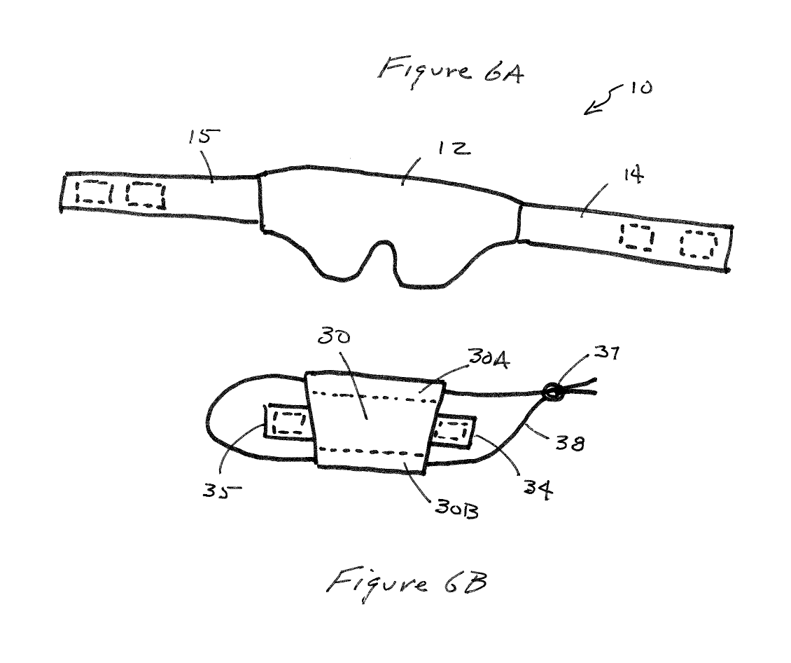

FIGS. 6A-B and 7 illustrate another embodiment of an accessory sleep mask device 10 in combination with a headrest seat attachment base 30 for securing about the headrest portion 37 of a seat 39. Referring to the FIG. 6A, an accessory device 10 comprises a head retention piece 12 having a pair of side bands 14 and 15 with one end secured thereto and extending in opposite directions outwardly therefrom, which may be similar to or different from the construction and function of head retention piece 12 described with reference to FIGS. 1-5. At least a portion of a retention piece 12 is intended for contact with the head of the wearer. The free ends of bands 14 and 15 have secured thereto one portion of a hook and pile type connector system, as illustrated in phantom.

Referring to the FIG. 6B, headrest seat attachment base 30 comprises a piece of fabric and having attached thereto attachment flaps 34 and 35, each of which has attached thereto the other of the section of the hook and pile type connector system not connected to the free ends of bands 14 and 15, respectively. The top and bottom ends of base 30 are folded back onto the base fabric on the rearward facing surface thereof to form passages 30A-B, as illustrated in phantom, and are configured to receive therein an elastic cord 38 and allow passage freely therethrough. Cord 38 may be threaded through a cord lock 37 which enables the cord 38 to be sized to form a loop.

In one embodiment, accessory device 10 in accordance with embodiments illustrated in FIGS. 6A-B and 7 may be provided as a kit with multiple cords 38 of various lengths which may be threaded through cord lock 37 and used to selectively secure base 30 to various headrest 37 of various configurations and dimensions.

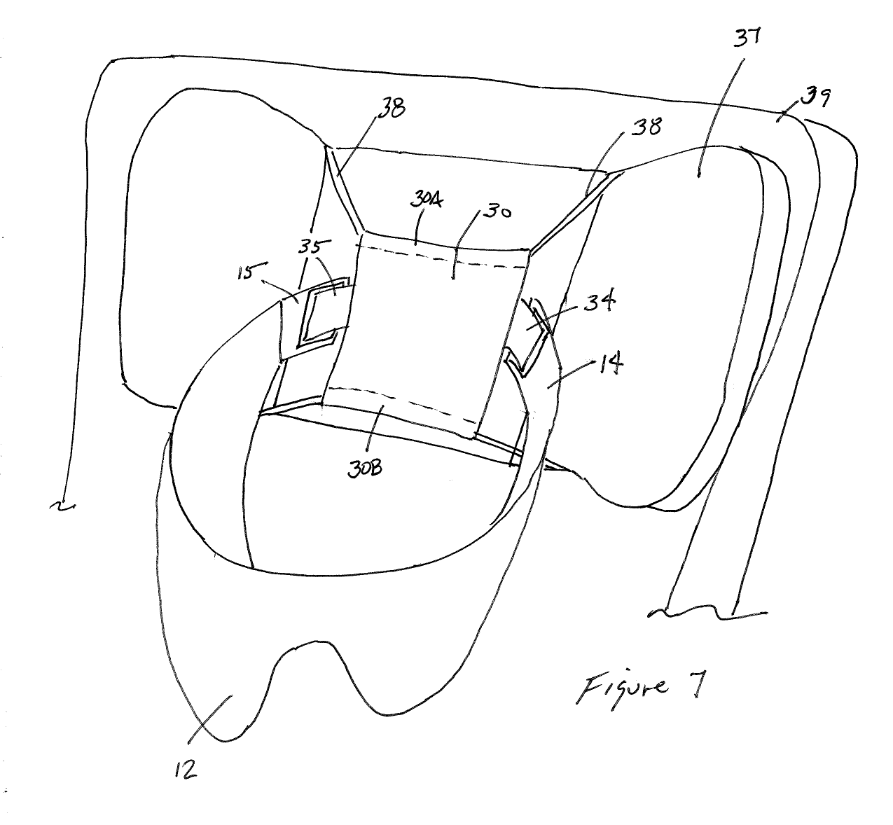

FIG. 7 illustrates conceptually a perspective view of the accessory device 10 embodiment of FIGS. 6A-B as attached to the headrest 37 of a seat 39. The portions of cord 38 extending outward from base 30 are stretched to fit around the side supports of a head headrest 37 of seat 39 and the size of the loop formed by cord 38 extending through base 30 adjusted accordingly utilizing cord lock 37, as necessary. With the head retention piece 12 at least partially worn by the subject (not shown), straps 14 and 15 are connected to attachment flaps 34 and 35, respectively, so that the complementary mating sections of the hook and pile type connector systems are secured to one another thereby securing head retention piece 12 to base 30. In embodiments, the length of straps 14 and 15 may vary first of

In all the foregoing embodiments, any known means may be used to secure the side band components of the accessory device to head retention piece including, but not limited to, hook and pile type connectors, snaps, buttons, buckles, loops, etc.

Further, the accessory retention device described herein may be packaged as a kit of the component parts, as described herein, which may be assembled by the user, or in conjunction with other items, such as a travel pillow to provide additional comfort for the occupant of the transportation seat.

In the contemplated embodiments, the side bands may be of variable length with adjustment mechanisms for selectively adjusting the appropriate length thereof for maximum user comfort. In addition, the side bands may be securable to the clasp mechanisms using any known mechanism such as buckles, clips, snaps, loops, Velcro, etc.

It will be obvious to those recently skilled in the art that modifications to the apparatus and process disclosed here in may occur, including substitution of various component values or nodes of connection, without parting from the true spirit and scope of the disclosure.

What is claimed is:

1. A head restraint apparatus comprising:
a head retention piece, and
a pair of width adjustable clasps secured to the head retention piece, each of the clasps being expandable to a width for temporarily securing the clasp about a surface having a thickness.

2. The head restraint apparatus of claim 1 further comprising at least one strap secured intermediate the head retention piece and one of the clasps.

3. The head restraint apparatus of claim 2 further comprising a pair of straps, each secured intermediate the head retention piece and one of the clasps.

4. A kit comprising an accessory device for use with transportation seats, the kit comprising:
a head retention piece, a portion of which is in contact with the wearer's head, and;
at least one strap removably securable to the head retention piece and about a transportation seat.

5. The kit of claim 4 further comprising:
a plurality of stretchable straps, each strap removably securable to the head retention piece and about a transportation seat.

6. The kit of claim 4 wherein the at least one stretchable strap has first and second ends removably securable to the head retention piece.

7. The kit of claim 5 wherein each of the plurality of stretchable straps has first and second ends removably securable to the head retention piece.

8. A head restraint kit for use with transportation seats comprising:
a head retention piece, and
a base portion adjustably securable about a surface having a thickness and further temporarily securable to the head retention piece.

9. The head restraint kit of claim 8 wherein the head retention piece comprises at least one strap securable to the base portion.

10. The head restraint kit of claim 8 wherein the head retention piece comprises a pair of straps securable to the base portion.

11. The head restraint kit of claim 8 wherein the pair of straps and the base contain perspective complementary hook and pile attachment mechanisms for removably securing the straps to the base portion.

12. The head restraint kit of claim 8 wherein base portion comprises a cord movably retain along to edges of the base portion to allow for free movement therethrough.

13. The head restraint kit of claim 12 wherein the cord is stretchable.

14. A method for securing a head restraint apparatus to a transportation seat, the method comprising:
providing a head restraint apparatus comprising a head retention piece having a pair of straps extending outward there from and a base portion adjustably securable about a surface having a thickness;
removably securing the base portion to the surface; and
removably securing the head restraint apparatus to the base portion.

15. The head restraint apparatus of claim 1 wherein the clasp width is greater than the surface thickness.
